(19) United States
(12) Patent Application Publication
(10) Pub. No.: US 2026/0295146 A1
(43) Pub. Date: Oct. 1, 2026

(54) FEMININE CARE DEVICE

(71) Applicant: Anita Nischal, Old Bridge, NJ (US)

(72) Inventor: Anita Nischal, Old Bridge, NJ (US)

(21) Appl. No.: 19/457,766

(22) Filed: Jan. 23, 2026

Related U.S. Application Data
(63) Continuation of application No. 17/977,553, filed on Oct. 31, 2022, now abandoned.
(60) Provisional application No. 63/274,243, filed on Nov. 1, 2021.

(51) Int. Cl. A61M 3/02; A46B 9/02; A46B 11/00; A61M 31/00

(52) U.S. Cl. CPC A61M 3/0279; A46B 9/026; A46B 11/0055; A61M 3/0262; A61M 31/00; A46B 2200/1006; A61M 2210/1475

(57) ABSTRACT

A feminine care device comprising a wand having a handle portion and an elongated stem portion. A cap is configured to overlay the elongated stem, where the cap has an open end, a closed end, and an outer cylindrical surface. A plurality of bristles is coupled to the closed end of the cap. The bristles are arranged along the outer cylindrical surface in a serpentine fashion.

### CROSS-REFERENCE TO RELATED APPLICATIONS

This application claims priority to U.S. Provisional Application No. 63/274,243, filed Nov. 1, 2021, which application is hereby incorporated in its entirety by reference.

### TECHNICAL FIELD

The present disclosure generally relates to a feminine care product, and more specifically, to a brush that removes excessive vaginal secretions.

### BACKGROUND

Women's vaginal health is a critical aspect of overall well-being and is closely linked to hormonal changes, menstrual cycles, sexual activity, and microbiological balance within the vaginal cavity. Normal vaginal discharge varies in appearance, consistency, and volume throughout the menstrual cycle; however, deviations from a woman's typical discharge pattern may signal disruptions in vaginal pH, bacterial imbalance, or infection. Despite the prevalence of vaginal discomfort and uncertainty, many women lack accessible tools to monitor, understand, and proactively manage their vaginal health outside of clinical settings.

Currently, women largely rely on subjective symptoms, over-the-counter products, or clinical examinations to assess vaginal health. Gynecological examinations allow trained professionals to visually and physically differentiate between normal discharge and pathological conditions, but such examinations are episodic, require appointments, and are not available for daily or preventative use. As a result, women frequently experience anxiety and uncertainty regarding whether vaginal discharge is normal, often leading to delayed care, unnecessary medical visits, or inappropriate self-treatment.

Additionally, disruptions to vaginal pH can cause minor discomfort, irritation, odor, and chronic wetness, which may be manageable through early intervention using simple home-based remedies if detected promptly. However, existing products—such as liquid douches and topical medications—are limited in effectiveness, may further disrupt the vaginal microbiome, and do not promote regular, informed self-awareness of vaginal conditions. Many healthcare providers discourage routine douching due to its association with adverse outcomes, leaving women with few safe and effective alternatives for internal vaginal maintenance.

A further limitation of current treatments involves bacterial biofilms within the vaginal cavity. Certain bacteria form protective biofilms that render them resistant to traditional medications, contributing to recurrent or chronic vaginal infections and prolonged discomfort. At present, there are no readily available treatments specifically designed to mechanically disrupt vaginal biofilms or to facilitate targeted delivery of biofilm-disrupting agents within the vaginal cavity in a non-clinical, user-controlled manner.

Accordingly, there exists a significant need for a device that enables women to safely monitor and manage vaginal discharge, gain intimate awareness of normal versus abnormal conditions, assist in restoring and maintaining vaginal pH balance, and facilitate the delivery of substances capable of disrupting vaginal biofilms. Such a device would empower women with greater control over their vaginal health, reduce uncertainty and insecurity related to discharge, and provide preventative and supportive care outside traditional clinical environments.

### SUMMARY

The present disclosure is directed to a feminine care device. In particular, this device is a brush that removes excessive vaginal secretions. It is directed to women of all ages, specifically targeting the onset of menarche and throughout their lives.

The feminine care device includes a wand having a handle portion and an elongated stem portion. A cap is configured to overlay the stem portion. The cap includes an open end, a closed end, and an outer cylindrical surface.

A plurality of bristles is coupled to the closed end of the cap. The bristles are arranged along the outer cylindrical surface in a serpentine fashion.

In some implementations, wherein the wand may be made of material having mechanical properties more rigid than the material comprising the cap. This provides a rigid internal core to provide structural stability and controlled manipulation of the device, while an external soft application surface is provided to allow gentle contact with vaginal tissue during use to minimize discomfort and reduce the risk of tissue irritation.

In some implementations, the wand is made from hard grade to medium hard grade silicone, such as one of shore A 60 to 80.

In some implementations, the cap is made from soft grade to medium soft grade silicone, such as one of shore A 10 to 55.

In some implementations, a second plurality of bristles may be affixed to a crown of the cap.

In some implementations, the second plurality of bristles are concentrically arranged about the perimeter of the cap.

In some implementations, the cap further comprises a plurality of serpentine-shaped grooves formed about an outer cylindrical surface of the cap, where the plurality of serpentine-shaped grooves cooperate with an application surface to provide a secondary scraping action that assists in dislodging and removing biological material from the vaginal cavity or other applied areas of the user as the user turns the device in the applied area.

In some implementations, the stem portion further comprises a proximal end and a distal end, where the proximal end is made from hard grade or medium hard grade silicone, such as one of shore A 60 to 80, and the distal end is made from soft grade to medium soft grade silicone, such as one of shore A 10 to 55.

In some implementations, the cap further comprises a cylindrical sleeve. The closed end and the cylindrical sleeve encapsulate the stem portion.

In some implementations, the plurality of bristles is made from soft grade to medium soft grade silicone, such as one of shore A 10 to 55.

In some implementations, the second plurality of bristles is made from soft grade to medium soft grade silicone, such as one of shore A 10 to 55.

The disclosure herein is a summary of the invention and not an extensive overview of all contemplated embodiments. It should be appreciated that many other features, applications, embodiments, implementations and/or variations of the disclosed technology will be apparent from the accompanying drawings and from the following detailed description. While multiple implementations are disclosed, the drawings and detailed description are to be regarded as illustrative in nature and not restrictive.

### BRIEF DESCRIPTION OF THE DRAWINGS

This disclosure may be better understood by referring to the following figures. The components in the figures are not necessarily to scale, emphasis instead being placed upon illustrating the principles of the disclosure. In the figures, the reference numerals designate corresponding parts throughout the different views.

FIG. 1 is a front view illustrating one example of a feminine care device according to the teachings of the present disclosure.

FIG. 2 is a front view illustrating one example of a wand of the feminine care device of FIG. 1.

FIG. 3 is a cross-section view of the wand of FIG. 2.

FIG. 4 is a top view of the wand of FIG. 2.

FIG. 5 is a cross-section view of the feminine care device of FIG. 1.

FIG. 6 is a bottom view illustrating one example of a cap of the feminine care device of FIG. 1.

FIG. 7 is a top view of the cap of FIG. 6.

### DETAILED DESCRIPTION

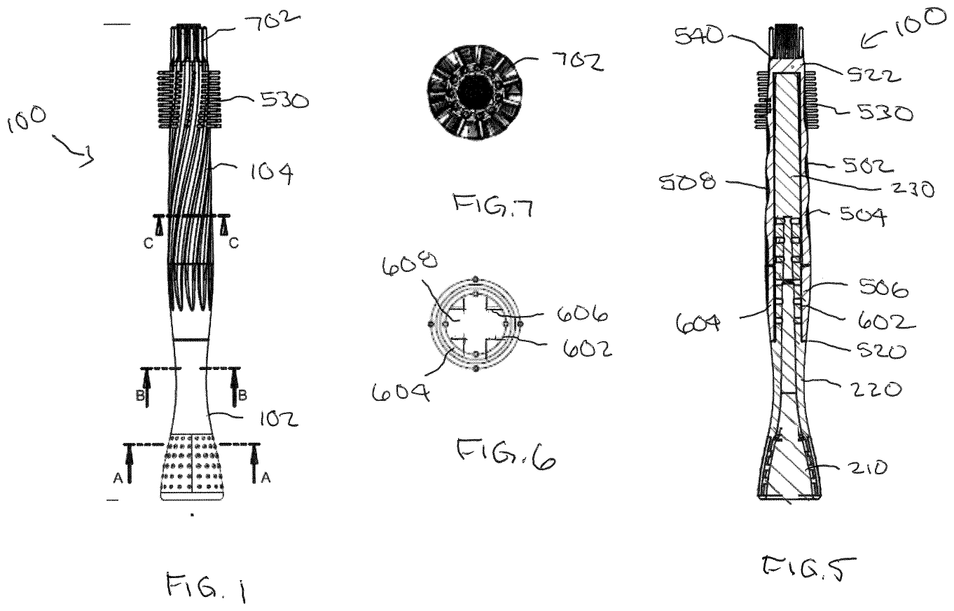

FIGS. 1-7 illustrates one example of an implementation of a feminine care device according to the teachings of the present disclosure. As described herein, the device is a cylindrical shaped hand-held unit with a comfortable grip and small tapered tip for ease of vaginal entry. Multiple small, short tapered smooth soft bristles cover a substantial portion of the cylinder. There is an ergonomic base of the cylinder for easy grip easy to rotate in the vaginal cavity.

FIG. 1 is a front view of one example of a feminine care device 100 according to the teachings of the present disclosure. As shown, the device 100 includes a wand 102 and a cap 104 coupled to a top portion thereof. The various components of the wand 102 and the cap 104 may be made of plastic, ceramic, polyvinyl chloride (PVC), medical-grade silicone for softness, or any other suitable medical-grade material.

FIG. 2 is a front view of the wand 102. As shown, the wand 102 comprises an elongated generally cylindrical shaped body 202 having a base 210, a handle portion 220, and an elongated stem portion 230. In preferred embodiments, the base 210, the handle portion 220, and the stem portion 230 may be integrally formed from hard grade to medium hard grade silicone, such as, for example, one of shore A 60 to 80. In other implementations, the base 210 and the handle portion 220 may be constructed from hard grade to medium hard grade silicone, while the stem portion 230 may be constructed from soft grade to medium soft grade silicone, such as, for example, one of shore A 10 to 55. In further implementations, the base 210, handle portion 220, and stem portion 230 may be integrally formed from any combination of hard grade to medium hard grade or soft grade to medium soft grade silicone. In other implementations, the base 210, handle portion 220, and stem portion 230 may be integrally formed from any combination of hard grade to medium hard grade or soft grade to medium soft grade silicone. In further implementations, the base 210 may comprise hard grade to medium hard grade silicone but be overlayed with soft grade to medium soft grade silicone to provide the user with a soft gripping surface.

In some implementations, the wand 102 may be constructed to a total length of between 4.5 to 6 inches.

FIG. 3 is a cross-sectional view of the wand 102. As better shown, the base 210 comprises a solid conical-shaped body 302 having a bottom 304 and a top 306.

The handle portion 220 may include hour-glass shaped body 312 having a bottom end 314 and a top end 316. The diametrical dimensions of the handle portion 220 may be greater than both the diametrical dimensions of the base 302 at the top 306 and the stem portion 230, such as to define a lower seat 315 and an upper seat 317.

The stem portion 230 may include an elongated solid body 320 having a proximal end 322 and a distal end 324. The proximal end 322 may comprise a generally cylindrical shape.

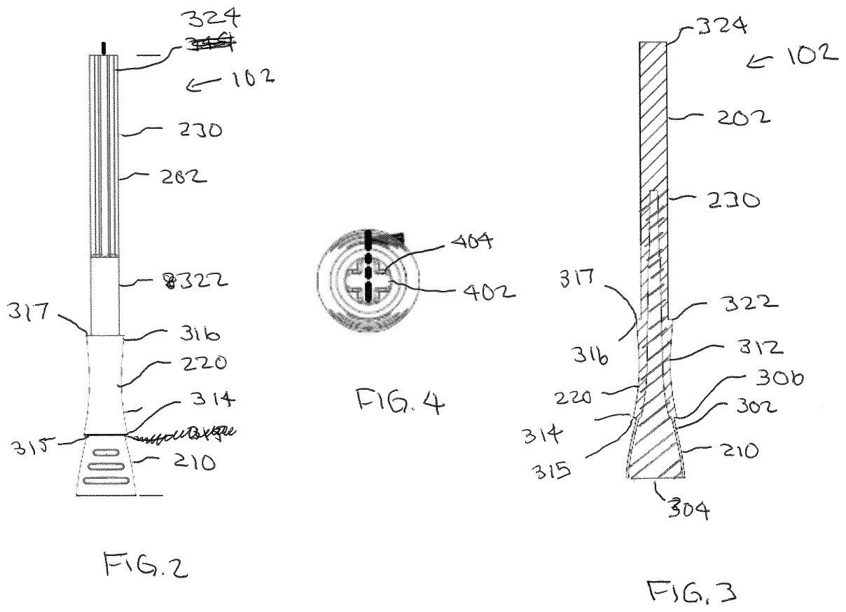

FIG. 4 is a top view of the wand 102. As shown, the distal end 324 may comprise a substantially cross-shaped cross-section 402. While shown as a cross-shaped cross-section, in other implementations, the cross-section 402 of the distal end 324 may comprise any other suitable geometrical shape.

As further shown, the cross-shaped cross-section 402 may include a plurality of substantially planar outer surfaces 404.

Referring back to FIG. 2, in some implementations, the proximal end 322 may be constructed from hard grade to medium hard grade silicone, while the distal end 324 may be constructed from soft grade to medium soft grade silicone. In other implementations, the proximal end 322 and the distal end 324 may be integrally formed from any combination of hard grade to medium hard grade or soft grade to medium soft grade silicone.

FIG. 5 is a cross-sectional view of feminine care device 100. Referring briefly back to FIG. 1, the cap 104 may comprise an elongated blunt-nosed body 110. In some implementations, the cap 104 may comprise a plurality of serpentine-shaped grooves 112 longitudinally formed along an outer cylindrical surface 114 of the cap. The serpentine-shaped grooves 112 cooperate with the application surface to provide a secondary scraping action that assists in dislodging and removing biological material from the vaginal cavity or other applied areas of the user as the user turns the device in the applied area.

Referring back to FIG. 5, the cap 104 may include an interior pocket 502. The interior pocket 502 and the outer cylindrical surface 114 define an annular sleeve 504 for receiving the stem portion 230 of the wand 104. As such, the cap 104 is adapted to overlay the stem portion 230.

The pocket 502 may include a first section 506 and a second section 508. The first section 506 may be configured to receive the proximal end 322 of the stem portion 230. As such, and particularly in the example show, the first section 506 of the pocket 502 may define an inner cylindrical sidewall 602 (FIG. 6) having a circular cross-section 604 (FIG. 6).

FIG. 6 is a bottom view of cap 104. As shown, the second section 508 may be configured to receive the distal end 324 of the stem portion 230. As such, and particularly in the example shown, the second section 508 of the pocket 502 may comprise a plurality of inner planar sidewalls 606 corresponding in shape with the planar outer surfaces 246 of the stem portion 230. The plurality of inner planar sidewalls 606 define a cavity 608 having a cross-shaped cross-section. The mechanical tolerance between the outer diameter of the proximal end 322 and the inner diameter of the cylindrical sidewall 602, and the planar outer surfaces 246 of the stem portion 230 and the inner planar sidewalls 606 of the cap, respectively, should be sufficient to allow the cape to slide over the entire the stem portion 230 of the wand 102.

In preferred implementations, the cap 104 may be constructed from soft grade to medium soft grade silicone. In other implementations, the cap 104 may be made from neoprene, rubber, or any suitable medical-grade material.

The cap 104 may further include an open end 520 and an opposing blunt-shaped, closed-end 522. The open end 520 of the cap 104 may be coupled to the handle portion 220 along seat 317 by bonding, glue, adhesive, or any other suitable means. In other implementations, the cap may be adhered to the stem portion 230 by bonding, glue, adhesive, or any other suitable means. Thus, the stem portion 230 is encapsulated within the cap 104. As such, the device 100 is configured with a rigid internal core to provide structural stability and controlled manipulation, while an external soft application surface is provided to allow gentle contact with vaginal tissue during use to minimize discomfort and reduce the risk of tissue irritation.

The closed end 522 may include a first set of bristles 530 affixed to and extending from an upper portion of the outer cylindrical wall 114 of the cap. As best shown in FIG. 1, the first set of bristles 530 may be arranged along the serpentine-shaped grooves 112 to better facilitate the removal of biological matter from the vaginal cavity of the user as the user rotates the wand 102. The first set of bristles 530 may be made of rubber, soft grade silicon or any other suitable medical-grade material. The first set of bristles 530 may each be, for example, 3 to 6 mm in length and configured with an appropriate degree of rigidity to facilitate the removal of residual biological material from the vaginal cavity of the user.

In other implementations, the closed end 522 may further include a crown 540 affixed to and extending from the top of the closed end 522. As shown in FIG. 7, the crown 540 may include a second set of bristles 702 concentrically arranged about the perimeter of the crown 522. The second set of bristles 702 may further aid in the removal of residual biological material from the vaginal cavity of the user. In some implementations, the second set of bristles 702 may be made of rubber, soft grade silicon or any other suitable medical-grade material. The second set of bristles 702 may each be, for example, 3 to 6 mm in length and configured with an appropriate degree of rigidity to facilitate the removal of residual biological material from the vaginal cavity of the user.

The device of the present disclosure is configured to facilitate the removal of excess vaginal discharge and residual biological material from the vaginal canal of a user. In various embodiments, the device may be used at the conclusion of the menstrual cycle to assist in removing residual menstrual blood that may contribute to vaginal odor. The device may further be used to remove residual vaginal medications, including suppositories, creams, and similar formulations.

In certain applications, the device may be utilized at the onset of vaginal discomfort associated with yeast or bacterial imbalance to assist in reducing conditions that may contribute to infection and discomfort. The device may also aid in alleviating vaginal discomfort during early symptom onset. Additionally, the device may serve as an adjunct to biofilm-disrupting applications, including the controlled application of oils or other topical agents.

The device may further be used in cases of vaginal dryness to apply natural lubricants, such as plant-based oils, including for postmenopausal users. By facilitating the removal of excess discharge, the device may assist in maintaining vaginal pH balance, reducing chronic vaginal wetness, and mitigating conditions associated with vaginal odor.

The vaginal brush comprising the bristles 530 of the present disclosure is intended to reduce or eliminate the need for liquid douching products, as it enables mechanical removal of pooled discharge from the vaginal vault using water alone. In use, the brush may be moistened with water, inserted into the vaginal canal, and rotated one or more times to remove excess discharge without the introduction of fragrances, chemical cleansers, or medicated solutions. Because only water contacts the vaginal cavity, the device is configured to minimize disruption to the natural vaginal microbiota and pH.

The softness and configuration of the bristles further permit gentle cleansing of the external vaginal anatomy, including the labial folds and surrounding crevices, providing improved hygiene compared to abrasive cloths or manual cleansing, and assisting in odor control.

It is preferred to package the device in a small case with power cord and UV light to prevent bacteria growth. Another option is to tote the device in a small cylinder case, resembling a lipstick carrying case.

It is recommended to use the device daily in the shower or in a private space where there is a sink to wet the device before insertion and wash the device after use. It is also recommended to replace the device monthly or after a certain number of uses.

The device may be easy to clean with soap and water, and air dried or towel dried before placing in the UV case. The device may remain in the shower between uses or can be portable in the same carrying case.

The various components of the device may also be made in various vibrant colors. All components of the device will be made from materials free of any chemical byproduct and safe for insertion into a body cavity.

In general, terms such as “coupled to,” and “configured for coupling to,” and “secured to,” and “configured for securing to” and “in communication with” (for example, a first component is “coupled to” or “is configured for coupling to” or is “configured for securing to” or is “in communication with” a second component) are used herein to indicate a structural, functional, mechanical, electrical, signal, optical, magnetic, electromagnetic, ionic or fluidic relationship between two or more components or elements. As such, the fact that one component is said to be in communication with a second component is not intended to exclude the possibility that additional components may be present between, and/or operatively associated or engaged with, the first and second components.

Although the previous description illustrates particular examples of various implementations, the present disclosure is not limited to the foregoing illustrative examples. A person skilled in the art is aware that the disclosure as defined by the appended claims and their equivalents can be applied in various further implementations and modifications. In particular, a combination of the various features of the described implementations is possible, as far as these features are not in contradiction with each other. Accordingly, the foregoing description of implementations has been presented for purposes of illustration and description. Modifications and variations are possible in light of the above description.

What is claimed is:

1. A feminine care device comprising:
a wand having a handle portion and an elongated stem portion;
a cap configured to overlay the elongated stem, the cap having an open end, a closed end, and an outer cylindrical surface; and
a plurality of bristles coupled to the closed end of the cap, the plurality of bristles being arranged along the outer cylindrical surface in a serpentine fashion.

2. The feminine care device of claim 1, wherein the wand is made of material having mechanical properties more rigid than the material comprising the cap, providing a rigid internal core to provide structural stability and controlled manipulation of the device, while an external soft application surface is provided to allow gentle contact with vaginal tissue during use to minimize discomfort and reduce the risk of tissue irritation.

3. The feminine care device of claim 1, wherein the wand is made from hard grade to medium hard grade silicone, such as one of shore A 60 to 80.

4. The feminine care device of claim 1, wherein the cap is made from soft grade to medium soft grade silicone, such as one of shore A 10 to 55.

5. The feminine care device of claim 1 further comprising a second set of bristles affixed to a crown of the cap.

6. The feminine care device of claim 1, wherein the second plurality of bristles are concentrically arranged about the perimeter of the cap.

7. The feminine care device of claim 1, wherein the cap further comprises a plurality of serpentine-shaped grooves formed about an outer cylindrical surface of the cap, where the plurality of serpentine-shaped grooves cooperate with an application surface to provide a secondary scraping action that assists in dislodging and removing biological material from the vaginal cavity or other applied areas of the user as the user turns the device in the applied area.

8. The feminine care device of claim 1, wherein the stem portion further comprises a proximal end and a distal end, where the proximal end is made from hard grade or medium hard grade silicone, such as one of shore A 60 to 80, and the distal end is made from soft grade to medium soft grade silicone, such as one of shore A 10 to 55.

9. The feminine care device of claim 1, wherein the cap further comprised a cylindrical sleeve, and the closed end and the cylindrical sleeve encapsulate the stem portion.

10. The feminine care device of claim 1, wherein the plurality of bristles is made from soft grade to medium soft grade silicone, such as one of shore A 10 to 55.

11. The feminine care device of claim 1, wherein the second plurality of bristles is made from soft grade to medium soft grade silicone, such as one of shore A 10 to 55.
